(19) United States
(12) Patent Application Publication
(10) Pub. No.: US 2019/0327945 A1
(43) Pub. Date: Oct. 31, 2019

(54) REGENERATION METHOD USING SOMATIC CELL NUCLEAR TRANSFER (SCNT) CELL AND BLASTOCYST COMPLEMENTATION

(71) Applicant: RIDDLE INSTITUTE FOR REGENERATIVE MEDICINE, Tomball, TX (US)

(72) Inventor: Michael C. Riddle, Jr., Tomball, TX (US)

(73) Assignee: RIDDLE INSTITUTE FOR REGENERATIVE MEDICINE, Tomball, TX (US)

(21) Appl. No.: 16/309,907

(22) PCT Filed: Jun. 14, 2017
(86) PCT No.: PCT/US17/37487

§ 371 Date: Dec. 13, 2018

Related U.S. Application Data
(60) Provisional application No. 62/350,005, filed on Jun. 14, 2016.

(51) Int. Cl. A01K 67/027

(52) U.S. Cl. CPC A01K 67/0271; A01K 67/0276; A01K 2217/05; A01K 2227/105; A01K 2267/025

(57) ABSTRACT

We disclose that a target organ such as kidney can be regenerated by complementing a developmental deficiency leading to a lack of development of the target organ in a non-human first mammal by injecting a somatic cell nuclear transfer cell (SCNT cell) into a developed blastocyst of the non-human first mammal. We also disclose a method for producing a target organ, using an SCNT cell, in a living body of a non-human first mammal having an abnormality associated with a lack of development of the target organ in a development stage, the target organ produced being derived from a second mammal that is an individual different from the non-human first mammal.

### BACKGROUND OF THE INVENTION

### Field of the Disclosure

Generally, the present disclosure relates to a method for producing a desired cell-derived organ in vivo using an SCNT cell.

### Description of the Related Art

In discussing regenerative medicine in the form of cell transplantation or organ transplantation, when an induced pluripotent stem cell (iPS cell) is injected into the inner space of a blastocyst, a resulting individual forms a chimeric mouse. There has been previously reported a rescue experiment of T-cell and B-cell lineages by blastocyst complementation, to which this technique is applied, the rescue experiment being carried out on a Rag-2 knockout mouse deficient in T-cell and B-cell lineages. This chimeric mouse assay is used as an in vivo assay system for verifying the differentiation of the T-cell lineage, for which no in vitro assay system is available.

Also, regarding regeneration of an organ, Nakauchi and coworkers (US 2011/0258715) report complementation of a deficiency of a pancreas in Pdx1^{LacZ/LacZ} mice by injection of iPS cells into a developed blastocyst, and further discovered that a transgenic animal having the pancreas thus complemented can transmit its phenotype to the next generation as a founder. These discoveries have revealed that organ regeneration can be accomplished by using such a founder.

However, the technique practiced by Nakauchi et al. requires induction of pleuripotency in somatic cells by culturing the somatic cells with reprogramming factors and establishing iPS cells therefrom. Selecting proper reprogramming factors, insuring induced pleuripotency of the putative iPS cells, and establishing the iPS cells represent opportunities for challenges to arise in implementing Nakauchi's work in the clinic for the improvement of human or animal health.

Other attempts to regenerate an organ have started with embryonic stem cells (ES cells) rather than iPS cells. However, ES cells from a first embryo of a given species will be genetically distinct from a second individual, even of the same species, that may desire a regenerated organ. Hence, transplant of a regenerated organ arising from an ES cell from a first embryo may require the second individual to undergo immunosuppressive therapy to prevent rejection of the regenerated organ, which would render the second individual immunocompromised, which is undesirable. Also, various parties may find it unethical to make use of ES cells from a first human embryo.

The present disclosure may address and/or at least reduce one or more of the problems identified above regarding the prior art and/or provide one or more of the desirable features listed above.

### SUMMARY OF THE DISCLOSURE

The following presents a simplified summary of the disclosure in order to provide a basic understanding of some aspects of the disclosure. This summary is not an exhaustive overview of the disclosure. It is not intended to identify key or critical elements of the disclosure or to delineate the scope of the disclosure. Its sole purpose is to present some concepts in a simplified form as a prelude to the more detailed description that is discussed later.

Generally, the present disclosure is directed to a technique for organ regeneration using a readily preparable somatic cell nuclear transfer cell (SCNT cell), the technique being suitable for industrial application. Specifically, the present disclosure may provide techniques for regenerating an “own organ” of an individual from one of the individual's somatic cells, such as skin, without the challenges that may arise from use of iPS cells.

### BRIEF DESCRIPTION OF THE DRAWINGS

The disclosure may be understood by reference to the following description taken in conjunction with the accompanying drawings, in which like reference numerals identify like elements, and in which:

FIG. 1A presents a schematic of a somatic cell nuclear transfer (SCNT) process for reproductive cloning, which is included herein as providing relevant background;

FIG. 1B presents a schematic of a somatic cell nuclear transfer (SCNT) process by which SCNT cells capable of blastocyst complementation may be produced, in accordance with embodiments herein;

FIG. 2 presents a schematic of blastocyst complementation using SCNT cells, in accordance with embodiments herein;

FIG. 3 provides a flowchart relating to a first method in accordance with embodiments herein; and

FIG. 4 provides a flowchart relating to a second method in accordance with embodiments herein.

While the subject matter disclosed herein is susceptible to various modifications and alternative forms, specific embodiments thereof have been shown by way of example in the drawings and are herein described in detail. It should be understood, however, that the description herein of specific embodiments is not intended to limit the disclosure to the particular forms disclosed, but on the contrary, the intention is to cover all modifications, equivalents, and alternatives falling within the spirit and scope of the disclosure as defined by the appended claims.

### DETAILED DESCRIPTION

Various illustrative embodiments of the disclosure are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present subject matter will now be described with reference to the attached figures. Various structures, systems and devices are schematically depicted in the drawings for purposes of explanation only and so as to not obscure the present disclosure with details that are well known to those skilled in the art. Nevertheless, the attached drawings are included to describe and explain illustrative examples of the present disclosure. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition will be expressly set forth in the specification in a definitional manner that directly and unequivocally provides the special definition for the term or phrase.

In one embodiment, in a blastocyst complementation method, a next generation is born when a deficiency of an organ, such as kidney, is complemented by injection of somatic cell nuclear transfer cells (SCNT cells) into a blastocyst, to yield a chimeric embryo, and that a transgenic animal having the kidney thus complemented can transmit its phenotype to the next generation as a founder. In other words, organ regeneration can be accomplished by using such a founder.

In one embodiment, the present disclosure provides a method for producing a target organ in a living body of a non-human first mammal having an abnormality associated with a lack of development of the target organ in a development stage, the target organ produced being derived from a second mammal that is an individual different from the non-human first mammal, the method comprising:

a) preparing an somatic cell nuclear transfer cell (SCNT cell) derived from the second mammal;

b) transplanting the cell into a blastocyst of the non-human first mammal, to yield a chimeric embryo;

c) developing the chimeric embryo in a uterus of a non-human third mammal to obtain at least one offspring comprising the target organ; and

d) obtaining the target organ from the at least one offspring.

In one embodiment, the SCNT cell is derived from one or more of a human, a rat, and a mouse.

In one embodiment, the SCNT cell is derived from one or more of a rat and a mouse.

In one embodiment, the organ to be produced is selected from a pancreas, a kidney, a thymus, and a hair.

In one embodiment, the non-human first mammal is a mouse.

In one embodiment, the mouse is one or more of a Sall 1 knockout mouse, a Pdx1-Hes1 transgenic mouse, a Pdx1 knockout mouse, and a nude mouse.

In one embodiment, the target organ is completely derived from the second mammal.

In one embodiment, the method may further comprise transferring a nucleus of a somatic cell of the second mammal into an enucleated oocyte, to obtain the SCNT cell. In one embodiment, the enucleated oocyte may be of the same species as the second mammal.

In one embodiment, the SCNT cell and the non-human first mammal may be in a xenogeneic relationship, i.e., the second mammal and the non-human first mammal may be from different species.

In one embodiment, the SCNT cell is derived from a rat, and the non-human first mammal is a mouse.

In another aspect, the present disclosure provides a non-human first mammal having an abnormality associated with a lack of development of a target organ in a development stage, the mammal being produced by a method including:

a) preparing an SCNT cell derived from a second mammal that is an individual different from the non-human first mammal;

b) transplanting the SCNT cell into a blastocyst of the non-human first mammal, to yield a chimeric embryo; and

c) developing the chimeric embryo in a uterus of a non-human third mammal to obtain at least one offspring comprising the target organ.

In another aspect, the present disclosure relates to use of a non-human first mammal having an abnormality associated with a lack of development of a target organ in a development stage, for production of the target organ using an SCNT cell.

In another aspect, the present disclosure provides a kit for producing a target organ, the kit comprising:

A) a non-human first mammal having an abnormality associated with a lack of development of the target organ in a development stage; and

B) an SCNT cell derived from a second mammal that is an individual different from the non-human first mammal.

In another aspect, the present disclosure provides a method for producing one or more of a target organ and a target body part, the method comprising:

A) providing an animal which includes a deficiency-responsible gene coding for a factor which causes a deficiency of one or more of an organ and a body part and gives one or more of no possibility of survival and difficulty in survival if the factor functions, and in which the one or more of an organ and a body part is complemented by blastocyst complementation, the deficiency-responsible gene coding for a factor which causes a deficiency of the one or more of a target organ and a target body part;

B) growing an ovum obtained from the animal into a blastocyst;

C) introducing a target SCNT cell into the blastocyst so as to produce a chimeric blastocyst, the target SCNT cell having a desired genome capable of complementing a deficiency caused by the deficiency-responsible gene; and

D) producing an individual from the chimeric blastocyst, and then obtaining the one or more of a target organ and a target body part from the individual.

In one embodiment, the method further comprises transferring a nucleus of a somatic cell of the second mammal into an enucleated oocyte, to obtain the SCNT cell. In one embodiment, the enucleated oocyte may be from the same species as the second mammal.

In one embodiment, the step D) includes developing the chimeric blastocyst in a uterus of a non-human third mammal to obtain at least one offspring comprising the target organ, and obtaining the target organ from the at least one offspring.

In another embodiment, the target SCNT cell is derived from one or more of a rat and a mouse.

In another embodiment, the one or more of a target organ and a target body part is selected from a pancreas, a kidney, a thymus, and a hair.

In still another embodiment, the animal is a mouse.

In another embodiment, the mouse is one or more of a Sall 1 knockout mouse, a Pdx1 knockout mouse, a Pdx1-Hes1 transgenic mouse, and a nude mouse.

In still another embodiment, the one or more of a target organ and a target body part is completely derived from the SCNT cell.

In still another embodiment, the SCNT cell and the non-human first mammal are in a xenogeneic relationship.

In still another embodiment, the SCNT cell is derived from a rat, and the non-human first mammal is a mouse.

In another aspect, the present disclosure provides a kit for producing one or more of a target organ and a target body part, the kit comprising:

A) a non-human animal which includes a gene coding for a factor which causes a deficiency of one or more of an organ and a body part and gives one or more of no possibility of survival and difficulty in survival if the factor functions, and in which the one or more of an organ and a body part is complemented by complement; and

B) an SCNT cell derived from a second mammal that is an individual different from the non-human first mammal.

In one embodiment, the non-human animal and the SCNT cell are in a xenogeneic relationship.

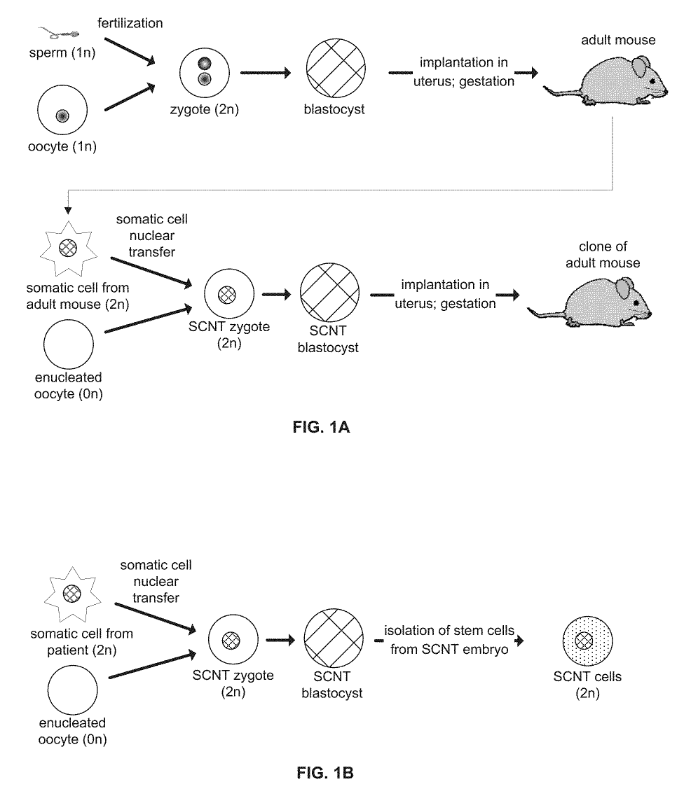

FIG. 1A provides general background regarding somatic cell nuclear transfer (SCNT) techniques in the context of so-called reproductive cloning. An adult mouse may be produced by fertilization of an oocyte (haploid or 1n) by a sperm (also haploid or 1n) to yield a zygote (diploid or 2n), followed by development of the zygote into a blastocyst, implantation thereof in the uterus of a female, gestation, birth, and post-natal development to yield the adult mouse. Fertilization and implantation may be performed by either conventional mating or in vitro fertilization (IVF) techniques. The adult mouse is genetically identical to the zygote.

Continuing through FIG. 1A, a diploid or 2n somatic cell (i.e., a non-gamete cell) of the adult mouse may be provided, e.g., by scrape of cells from skin or the buccal cavity (inner cheek) of the adult mouse. The nucleus of the somatic cell may be isolated using known techniques. Similarly, an oocyte may be provided and its nucleus removed, also by known techniques. The somatic cell nucleus may then be transferred into the enucleated oocyte, to yield an SCNT zygote. The SCNT zygote is genetically identical to (i.e., a clone of) the adult mouse. The SCNT zygote may develop into an SCNT blastocyst, and may be implanted in the uterus of a female by IVF techniques. The SCNT blastocyst may develop during gestation, undergo birth, and develop post-natally into a mature individual that is a clone of the adult mouse. The techniques required for each step are generally well known.

FIG. 1B schematically depicts SCNT techniques used in accordance with embodiments herein. A somatic cell (diploid, 2n) from a patient, e.g., a patient for whom it is desired to regenerate a target organ, is provided. The somatic cell's nucleus is isolated and transferred into an enucleated oocyte to yield an SCNT zygote. The SCNT zygote is genetically identical to (i.e., a clone of) the patient. The SCNT zygote may develop into an SCNT blastocyst and SCNT embryo. Rather than implant the SCNT blastocyst or embryo in the uterus of a female, the SCNT blastocyst or embryo may be retained in vitro, under conditions where gestational development cannot occur. However, even in vitro, stem cells may develop in the SCNT blastocyst or embryo. The stem cells may be isolated from the SCNT blastocyst or embryo, to yield SCNT cells which may be usable in accordance with embodiments herein.

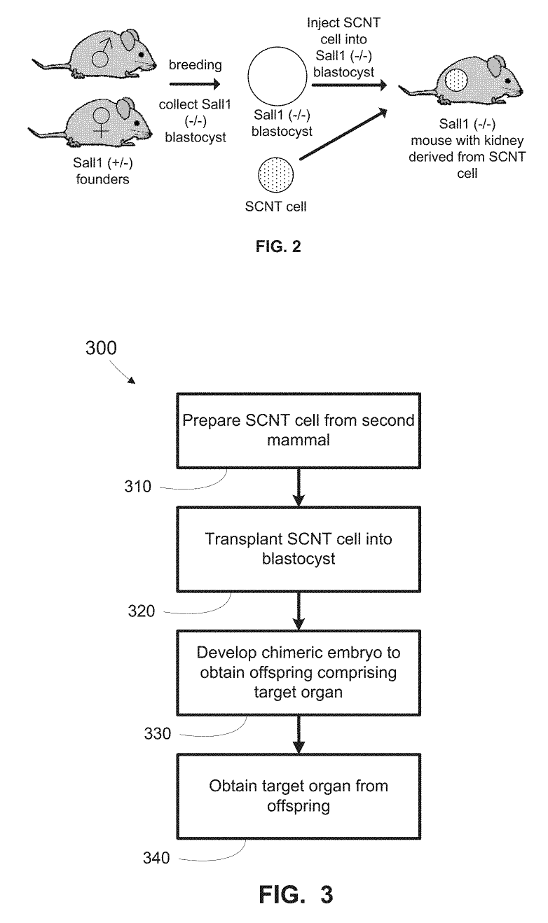

FIG. 2 schematically depicts further techniques, whereby an animal, e.g., a non-human mammal (e.g., in the depicted embodiment, a mouse) may be produced which comprises an organ (e.g., in the depicted embodiment, a kidney) derived from a second mammal. For example, a male and a female which are each heterozygous at the Sall1 locus (Sall1 (+/−)) may be bred and blastocysts recovered. Approximately ¼ of recovered blastocysts will have the Sall 1 (−/−) genotype, which results in a phenotype whereby an adult mouse lacks kidneys. However, if Sall1 (−/−) blastocysts receive SCNT cells (such as may be produced by techniques depicted in FIG. 1B), the SCNT cells (presuming they are Sall 1 (+/−), Sall 1 (+/+), or have a comparable genotype whereby kidney development may proceed normally) may give rise to a kidney in the adult Sall 1 (−/−) mouse.

Although FIG. 2 relates to mice and the Sall1 locus, the person of ordinary skill in the art having the benefit of the present disclosure will be aware that the techniques used would be applicable regarding other loci in mice or any locus in other animals.

In another embodiment, a homozygous Sall1 (−/−) embryo may be prepared without the need to breed sexually mature Sall1 (+/−) heterozygous animals, by use of so-called “gene drive” (e.g., CRISPR) techniques). For example, a CRISPR technique may be used to genetically modify oocytes and sperm to be Sall1 (−), in vitro fertilization techniques may then yield a homozygous Sall1 (−/−) embryo.

FIG. 3 presents a flowchart of a first method 300 in accordance with embodiments herein. The method 300 may comprise preparing (at 310) a somatic cell nuclear transfer cell (SCNT cell) derived from a second mammal; transplanting (at 320) the SCNT cell into a blastocyst of a non-human first mammal, to yield a chimeric embryo; developing (at 330) the chimeric embryo in a uterus of a non-human third mammal to obtain at least one offspring comprising a target organ; and obtaining (at 340) the target organ from the at least one offspring. By performing the method 300, the target organ may be produced in a living body of the non-human first mammal having an abnormality associated with a lack of development of the target organ in a development stage, the target organ produced being derived from the second mammal that is an individual different from the non-human first mammal.

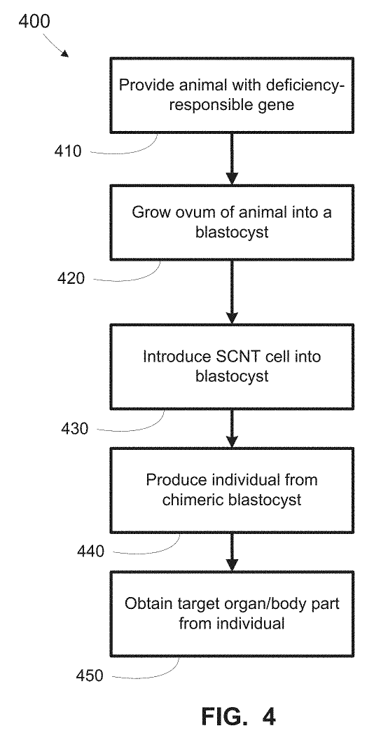

FIG. 4 provides a flowchart of a second method 400 in accordance with embodiments herein. As depicted, the method 400 may comprise providing (at 410) a non-human animal which includes a deficiency-responsible gene coding for a factor which causes a deficiency of one or more of an organ and a body part and gives one or more of no possibility of survival and difficulty in survival if the factor functions, and in which the one or more of an organ and a body part is complemented by blastocyst complementation, the deficiency-responsible gene coding for a factor which causes a deficiency of the one or more of a target organ and a target body part.

The method 400 may also comprise growing (at 420) an ovum obtained from the non-human animal into a blastocyst. The method 400 may further comprise introducing (at 430) a target somatic cell nuclear transfer cell (SCNT cell) into the blastocyst so as to produce a chimeric blastocyst, the target SCNT cell having a desired genome capable of complementing a deficiency caused by the deficiency-responsible gene. The method 400 may also comprise producing (at 440) an individual from the chimeric blastocyst. The method 400 may additionally comprise obtaining (at 450) the one or more of a target organ and a target body part from the individual.

The method 400 may be used to produce one or more of the target organ or the target body part.

In the present disclosure, SCNT cells to be transplanted in are prepared in accordance with the species of an animal for the organ to be produced. For example, when a human organ is to be produced, SCNT cells comprising a nucleus derived from a somatic cell of a human are prepared. When an organ of a mammal other than human is to be produced, cells derived from the mammal are prepared.

The organ to be produced in the method of the present disclosure may be any solid organ with a fixed shape, such as kidney, heart, pancreas, cerebellum, lung, thyroid gland, hair, and thymus. Preferable examples thereof include kidney, pancreas, hair, and thymus. Such solid organs are produced in the body of at least one offspring comprising the target organ by developing SCNT cells within a chimeric embryo that serves as a recipient. The SCNT cells can form all kinds of organs by being developed in an embryo. Accordingly, there is no limitation to the solid organ that can be produced depending on the kind of the SCNT cells to be used.

Meanwhile, the present disclosure is characterized in that an organ derived only from the transplanted cells is formed in the body of at least one offspring comprising the target organ individual derived from non-human embryo that serves as a recipient. Thus, it is not desirable to have a chimeric cell composition of the transplanted cells and the cells derived from the recipient non-human embryo. Therefore, as the recipient non-human embryo, it is desirable to use an embryo derived from an animal which has an abnormality associated with a lack of development of the organ to be produced in a development stage, and whose offspring has a deficiency of the organ. As long as the animal develops such an organ deficiency, a knockout animal having an organ deficiency as a result of the deficiency of a specific gene or a transgenic animal having an organ deficiency as a result of incorporating a specific gene may be used. Alternatively, a “founder” animal described herein may be used.

In one embodiment, when a kidney is produced as the organ, embryos of a Sall 1 knockout animal having an abnormality associated with a lack of development of a kidney in the development stage (Nishinakamura, R. et al., Development, Vol. 128, p. 3105-3115, 2001), or the like, can be used as the recipient non-human embryo. Meanwhile, when a pancreas is produced as the organ, embryos of a Pdx1 knockout animal having an abnormality associated with a lack of development of a pancreas in the development stage (Offield, M. F., et al., Development, Vol. 122, p. 983-995, 1996) can be used as the recipient non-human embryo. When a cerebellum is produced as the organ, embryos of a Wnt-1 (int-1) knockout animal having an abnormality associated with a lack of development of a cerebellum in the development stage (McMahon, A. P. and Bradley, A., Cell, Vol. 62, p. 1073-1085, 1990) can be used as the recipient non-human embryo. When a lung and a thyroid gland are produced as the organ, embryos of a T/ebp knockout animal having an abnormality associated with a lack of development of a lung and a thyroid gland in the development stage (Kimura, S., et al., Genes and Development, Vol. 10, p. 60-69, 1996), or the like, can be used as the recipient non-human embryo. Moreover, embryos of a dominant negative-type transgenic mutant animal model (Celli, G., et al., EMBO J., Vol. 17 pp. 1642-655, 1998) which overexpresses the deficiency of an intracellular domain of fibroblast growth factor (FGF) receptor (FGFR), and which causes deficiencies of multiple organs such as kidney and lung, can be used. Alternatively, nude mice can be used for production of hair or thymus.

In the present disclosure, the non-human animal derived from the recipient embryo may be any animal other than human, such as pig, rat, mouse, cattle, sheep, goat, horse, dog, baboon, chimpanzee, gorilla, orangutan, monkey, marmoset, and bonobo. It is preferable to collect embryos from a non-human animal having a similar adult size to that of the animal species for the organ to be produced.

Meanwhile, a mammal serving as the origin of the cell that is transplanted into a recipient blastocyst and that is for formation of the organ to be produced may be either human or a mammal other than human, such as, for example, pig, rat, mouse, cattle, sheep, goat, horse, dog, baboon, chimpanzee, gorilla, orangutan, monkey, marmoset, and bonobo.

The relationship between the recipient embryo and the SCNT cell to be transplanted may be an allogeneic (same species) relationship or a xenogeneic (different species) relationship. By transplanting the SCNT cell, prepared as described above, into the inner space of the recipient blastocyst, a chimeric mixture of the blastocyst-derived inner cell and the transplanted SCNT cell may be formed in the inner space of the blastocyst.

The blastocyst or embryo having an SCNT cell transplanted as described above may be transplanted into a uterus of a surrogate parent, such as a pseudo-pregnant or pregnant female animal of the species from which the blastocyst is derived. The blastocyst may develop in the uterus of the surrogate parent to obtain at least one offspring comprising the target organ. Then, the target organ can be obtained as a mammal cell-derived target organ from this litter.

According to the present disclosure, a technique for organ regeneration is provided, the technique being suitable for industrial application. This also provides a technique for regenerating an “own organ” from a somatic cell, such a skin, depending on the circumstance of an individual. The benefit to human health provided by a transplantable organ that may be genetically identical to the organ's recipient will be apparent to the person of ordinary skill in the art.

Moreover, it becomes possible to conduct research and development using organs derived from various genomes, the organs being provided by carrying out the present disclosure by way of producing an SCNT cell from a cell having a target genome.

In order to specifically describe embodiments of the present disclosure, exemplary embodiments will be described hereinafter. As an example, a method for producing a kidney derived from a mammal cell in a living body of a mouse will be described hereinbelow. It is understood that a pancreas, a hair, and a thymus can also be produced by such a method.

### (Non-Human Animal)

In order to produce a kidney derived from a cell of a mammal other than human in a living body of an animal such as a mouse, an animal such as a mouse having an abnormality associated with a lack of development of the kidney in a development stage may be prepared. In one embodiment of the present disclosure, a Sall 1 knockout mouse (Nishinakamura, R. et al., Development, Vol. 128, p. 3105-3115, 2001) can be used as the mouse having an abnormality associated with a lack of development of the kidney in a development stage. If this animal has a homozygous knockout genotype of Sall1 (−/−), the animal is characterized in that the kidney does not develop, and at least one offspring has no kidney. Alternatively, a founder animal described herein can also be used.

This mouse has no kidney formed and cannot survive if the deficiency of Sall1 gene is in a homozygous state (Sall1 (−/−)). Thus, the deficiency of Sall1 gene is maintained in a heterozygous state (Sall1 (+/−)). Mice each in the heterozygous state are bred with each other (Sall 1 (+/−).times.Sall1 (+/−)), and fertilized eggs are collected from the uterus. The fertilized eggs develop at a probability ratio of Sall 1 (+/+):Sall1 (+/−):Sall1 (−/−)=1:2:1, in terms of probability. In the present disclosure, an embryo of Sall1 (−/−), which develops at a probability of 25%, is used. However, it is difficult to determine the genotype in the stage of early embryo, and thus, it is practical to determine the genotype of at least one offspring comprising the target organ after birth and to use only individuals having the desired genotype of Sall1 (−/−) in the subsequent steps.

This knockout mouse may have the Sall1 gene knocked out in the preparation stage and have a gene of a fluorescent protein for detection, or green fluorescent protein (GFP), knocked in into the Sall1 gene region in an expressible state (Takasato, M. et al., Mechanisms of Development, Vol. 121, p. 547-557, 2004). When the regulatory region of this gene is activated by knocking-in such a fluorescent protein, expression of GFP occurs instead of Sall1, and the deficiency state of the Sall1 gene can be determined by fluorescence detection.

Further, the relationship between a recipient embryo and a cell to be transplanted in the present disclosure may be an allogeneic relationship or a xenogeneic relationship. There have been hitherto a large number of reports on the preparation of a chimeric animal in such a xenogeneic relationship in the art. For example, there have been actually reported about blastular chimeric animals between closely related animal species, such as the preparation of a chimera between rat and mouse (Mulnard, J. G., C. R. Acad. Sci. Paris. 276, 379-381 (1973); Stern, M. S., Nature. 243, 472-473 (1973); Tachi, S. & Tachi, C. Dev. Biol. 80, 18-27 (1980); Zeilmarker, G., Nature, 242, 115-116 (1973)), and the preparation of a chimera between sheep and goat (Fehilly, C. B., et al., Nature, 307, 634-636 (1984)). Therefore, in the present disclosure, for example, in the case of preparing a kidney derived from a cell of a mammal other than human in a living body of a mouse, a certain xenogeneic organ may be prepared in a recipient embryo based on these conventionally-known chimera creation methods (for example, a method of inserting cells to be transplanted into a recipient blastocyst (Fehilly, C. B., et al., Nature, 307, 634-636 (1984)).

The term “non-human first mammal” as used herein refers to a counterpart mammal from which a chimeric animal, a chimeric embryo, a chimeric blastocyst, or the like is produced using an SCNT cell.

The term “second mammal” as used herein refers to any mammal that is an individual different from the non-human first mammal, and may be an allogeneic individual or a xenogeneic one.

The term “non-human third mammal” as used herein refers to a mammal in which a chimeric embryo formed by transplanting a cell derived from a second mammal that is an individual different from a non-human first mammal into the blastocyst is developed in a uterus of the non-human third mammal (serving as a surrogate parent).

Note that although the terms “non-human first mammal” and “non-human third mammal” are sometimes referred to as a “non-human host mammal” or “host,” the “non-human first mammal” and the “non-human third mammal” may be animals different from each other. In the context of the present disclosure, it should be understood that which is indicated is apparent to those skilled in the art.

When a pancreas is produced as the organ, embryos of a Pdx1 knockout animal having an abnormality associated with a lack of development of pancreas in a development stage (Offield, M. F., et al., Development, Vol. 122, p. 983-995, 1996) or a founder animal described herein can be used as the recipient non-human embryo.

When a hair is produced as the organ, embryos of a hairless nude mouse can be used as the recipient non-human embryo.

When a thymus is produced as the organ, embryos of a nude mouse can be used as the recipient non-human embryo.

(Cell to be Transplanted)

Next, a cell to be transplanted into, for example, a kidney will be described. In order to produce a kidney derived from a mammal cell, an SCNT cell is prepared. With respect to the Sall 1 gene, the cell has a wild type genotype (Sall 1 (+/+)), and has an ability to develop into all kinds of cells in the kidney.

In one embodiment, this cell may further incorporate a fluorescence protein for specific detection in an expressible state prior to transplantation. For example, as a fluorescent protein used for such detection, the sequence of DsRed. T4 (Bevis B. J. and Glick B. S., Nature Biotechnology Vol. 20, p. 83-87, 2002), which is a DsRed genetic mutant, may be designed so as to be expressed in organs of almost the entire body under the control of a CAG promoter (cytomegalovirus enhancer and chicken actin gene promoter), and then be incorporated into an SCNT cell by electroporation. A fluorescence protein known in the art, such as a green fluorescence protein (GFP), may be used. By performing a fluorescent labeling on such a cell for transplantation, it can be easily detected whether or not a produced organ is composed of transplanted cells only.

The SCNT cell is transplanted into the inner space of a blastocyst having the aforementioned genotype of Sall1 (−/−) to prepare a blastocyst having a chimeric inner cell mass. This blastocyst having a chimeric inner cell mass is developed in a uterus of a surrogate parent to obtain at least one offspring comprising the target organ. In the case of using an SCNT cell which does not express marker such as GFP, the cell cannot be distinguished from the embryos of the host when used in the production of chimera, and it cannot be discriminated whether the complementation of the organ has been achieved. Therefore, in order to solve the problem, a fluorescent dye can be introduced into this cell line, thereby being capable of carrying out an experiment with the same protocol as those described in Examples and the like.

### (Method for Producing Founder Animal for Reproduction).

A founder animal for reproduction, used in the present disclosure, has the following characteristics: the animal includes a gene coding for a factor which causes a deficiency of one or more of an organ and a body part and gives one or more of no possibility of survival and difficulty in survival if the factor functions, and in which the one or more of an organ and a body part is complemented by blastocyst complementation. By producing a next generation animal using this animal (also referred to as a “founder animal” herein), it is possible to cause a target organ to be deficient, and to produce an organ having a desired genome type regarding the deficient organ. Moreover, it has been revealed that production using this method enables organ production in the next generation as well and also that the method can be used with SCNT cells.

The term “one or more of an organ and a body part, giving one or more of no possibility of survival and difficulty in survival if the factor functions” as used herein refers to, in regard to a certain factor, one that gives one or more of no possibility of survival and difficulty in survival when the factor causes the one or more of an organ and a body part to be deficient or dysfunctional (for example, to be not normal). For example, in the case of a foreign gene, when the gene is introduced into an animal and expressed normally, a deficiency occurs in a certain organ or body part, resulting in the animal being incapable of survival or having difficulty in survival. Difficulty in survival includes incapability of procreation of the next generation, and difficulty in the social life in a case of human. Such an organ or body part may be, for example, pancreas, liver, hair, thymus, or the like, but is not limited thereto.

Examples of genes involved in such events include Pdx-1 (for pancreas), Sall1 (for kidney), and the like.

Incidentally, to be used for organ regeneration, a gene should be selected with which an organ can be complemented and a resulting litter does not die after birth due to other factors (being incapable of ingesting milk from a mother mouse, for example). One example of such a gene is Sall 1. By using a gene possessing such properties, the disclosure of the present application can be carried out. In addition, even with the same phenotype of, for example, pancreatic deficiency, significance largely varies. Specifically, a knockout individual has a feature of improving productivity, while a transgenic individual has a feature of enabling clonal analysis of a lethal phenotype in addition to the feature of improving productivity.

The term “giving one or more of no possibility of survival and difficulty in survival if the factor functions” as used herein refers to, regarding a certain factor, a condition in which, if the factor functions, an animal as a host cannot survive at all and dies, or can survive but is substantially impossible to survive later due to reasons, such as difficulties in growth and reproduction. The term can be understood by using ordinary knowledge in the art.

The term “organ” as used herein is used to have an ordinary meaning in the art, and refers to organs constituting animal viscera in general.

The term “body part” as used herein refers to any part of a body, and also includes ones which are not generally referred to as organs. For example, when a kidney is taken as an example, a complete kidney is created when genes are normal. However, when some gene is deficient or has an abnormality, although an organ like a kidney may be created, a part of the organ may have an abnormality or deficiency. The part having such an abnormality or deficiency can be said to be an example of this “body part.” Gene defect or abnormality does not necessarily correspond to each organ, and it frequently occurs that a part thereof is affected. Accordingly, when a correspondence relationship to a gene is to be considered, it may be better to consider correspondence to a body part. Therefore, such a correspondence relationship is also taken into consideration herein.

The term “blastocyst complementation” as used herein refers to a technique for complementing a defective organ or body part by using the phenomenon in which a resulting individual obtained from injection of e.g. SCNT cells into an inner space of a blastocyst forms a chimeric embryo (and, upon normal pre- and post-natal development, a chimeric adult). A mammalian organ, such as kidney, pancreas, hair, and thymus, having a complicated cellular constitution formed of multiple kinds of cells can be produced in the living body of an animal, particularly, a non-human animal.

The term “label” as used herein may be any factor as long as it is used for distinguishing a complemented organ. For example, by causing a specific gene (such as, for example, a gene for expressing a fluorescence protein) to be expressed only in an organ to be complemented, the organ to be complemented can be distinguished from a host of complementation by a property (for example, fluorescence) derived from the specific gene. As described above, it can be distinguished whether an animal became complete by complementation with cells derived from exogenous cells or an animal became complete by complementation with cells derived from endogenous cells. Thus, it is possible to select a founder animal used in the present disclosure more easily. These cells may incorporate a fluorescence protein for specific detection in an expressible state prior to transplantation. For example, as a fluorescent protein used for such detection, the sequence of DsRed. T4 (Bevis B. J. and Glick B. S., Nature Biotechnology Vol. 20, p. 83-87, 2002), which is a DsRed genetic mutant, may be designed so as to be expressed in organs of almost the entire body under the control of a CAG promoter (cytomegalovirus enhancer and chicken actin gene promoter), and then be incorporated into an SCNT cell by electroporation. By performing a fluorescent labeling on such a cell for transplantation, it can be easily detected whether or not a produced organ is composed of transplanted cells only.

Examples of such label include: green fluorescent protein (GFP) genes; red fluorescent proteins (RFP); cyan fluorescent proteins (CFP); other fluorescent proteins; LacZ; and the like. In one embodiment, a method for producing a founder animal used in the present disclosure may comprise: A) providing a first zygote having a gene; B) growing the first zygote into a blastocyst; C) introducing an SCNT cell into the blastocyst so as to produce a chimeric embryo, the SCNT cell having an ability to complement a deficiency caused by the gene; and D) producing individuals from the chimeric blastocyst, and then selecting an individual in which the one or more of an organ and a part thereof has been complemented by the SCNT cell.

The terms “(deficiency-responsible) gene coding for a factor which causes a deficiency of one or more of an organ and a body part and gives one or more of no possibility of survival and difficulty in survival if the factor functions” and “deficiency-responsible gene” as used herein are used interchangeably and refers to, in regard to a certain gene, a gene that gives one or more of no possibility of survival and difficulty in survival when the factor functions (for example, in the case of a foreign gene, when the gene is introduced and expressed; in the case of an intrinsic gene, when such a gene is exposed to a condition in which the gene functions; or other cases) to cause the one or more of an organ and a body part to be deficient or dysfunctional (for example, to be not normal).

An SCNT cell may be referred to herein as a “pluripotent cell”.

The term “having an ability to complement a deficiency” as used herein refers to, in regard to a factor, gene, or the like, an ability capable of complementing an organ or a body part. The term “chimeric blastocyst” or “chimeric embryo” as used herein refers to a blastocyst or embryo comprising an SCNT cell, being in a chimeric state. Such a chimeric blastocyst or embryo can be produced by, in addition to an injection method, using a method such as a so-called “agglutination method” in which embryo+embryo, or embryo+cell are closely attached to each other in a Petri dish to produce a chimeric blastocyst. Further, the relationship between a recipient blastocyst or embryo and a cell to be transplanted in the present disclosure may be an allogeneic relationship or a xenogeneic relationship.

In a method for producing a founder animal in accordance with embodiments herein, the step of growing a blastocyst can be carried out by any publicly-known method for growing an oocyte or zygote into a blastocyst. The conditions for this are well known in the art, and described in Manipulating the Mouse Embryo, A LABORATORY MANUAL 3.sup.rd Edition 2002 (Cold Spring Harbor Laboratory Press, Cold Spring Harbor, N.Y.) (incorporated herein by reference).

In a method for producing a founder animal in accordance with embodiments herein, introducing an somatic cell nuclear transfer cell (SCNT cell) having an ability to complement a deficiency caused by the gene, into the blastocyst so as to produce a chimeric blastocyst may adopt any publicly-known method in the art as long as the somatic cell nuclear transfer cell (SCNT cell) can be introduced into the blastocyst. Examples of such a method include an injection method and agglutination; however, the method is not limited to these.

In a method for producing a founder animal in accordance with embodiments herein, a method for producing individuals from the chimeric blastocyst may adopt a publicly-known technique in the art. Generally, the chimeric blastocyst may be returned to a surrogate parent, and then pseudo-pregnancy of the surrogate parent may be caused so as to grow resulting individuals in the uterus of the surrogate parent. However, the method is not limited to this technique.

In a method for producing a founder animal in accordance with embodiments herein, selecting an individual in which the one or more of an organ and a body part thereof complemented can be carried out by using any technique allowing confirmation of complementation of the organ or body part.

An example thereof is identifying an identifier derived from the somatic cell nuclear transfer cell (SCNT cell). The term “identifier” as used herein refers to any factor which allows specifying of a certain individual, species, or the like, and identifying of the origin thereof, and is also referred to as “ID” in its abbreviation. Such an identifier could be, for example, a genomic sequence, phenotype, or the like unique to the somatic cell nuclear transfer cell (SCNT cell).

Alternatively, regarding such selecting, by using an SCNT cell which is labeled/marked or can be labeled/marked (including by gene expression), the selecting in the method for producing a founder animal may be carried out by identifying the label. In addition, it is understood that those in the art can carry out the selecting by modifying this technique as necessary.

### (Method of Organ Regeneration Using Founder Animal)

In another aspect, the present disclosure provides a method for producing one or more of a target organ and a target body part using a founder animal and using an somatic cell nuclear transfer cell (SCNT cell). The method comprises: providing a founder animal, in which a deficiency-responsible gene codes for a factor which causes a deficiency of the one or more of a target organ and a target body part; growing an ovum obtained from the animal into an blastocyst; introducing an somatic cell nuclear transfer cell (SCNT cell) into the blastocyst, to yield a chimeric embryo; and producing an individual from the chimeric blastocyst, and then obtaining the one or more of a target organ and a body part from the individual. The SCNT cell may have a desired genome capable of complementing a deficiency caused by the gene.

Developing the chimeric blastocyst may be carried out in a uterus of a non-human third mammal to obtain at least one offspring comprising the target organ, and obtaining the target organ from the at least one offspring.

(Formation of Pancreas)

The formation of a pancreas can be investigated by performing macroscopic or microscopic morphological analysis, gene expression analysis, and the like, using methods, such as visual inspection, microscopic observation after staining, and observation using fluorescence. For example, by performing visual inspection, the actual presence or absence of the organ, and features of the organ, such as the external appearance, can be investigated. Together with such a macroscopic morphological analysis, a tissue obtained after general tissue staining, such as hematoxylin-eosin staining, may be observed microscopically using a microscope. Such microscopic observation allows investigations to be performed, even on various concrete cellular compositions within the pancreas.

Furthermore, the gene expression analysis using fluorescence in such a way as to emit fluorescence according to conditions may also be performed. For example, a knockout mouse obtained through Pdx1-Lac-Z knock-in may have the following characteristics. When a fluorescent-labeled ES cell is used in a wild type (+/+) or heterozygous (+/−) individual, mottled fluorescence in a chimeric state is shown even though the contribution of the ES cell is observed.

On the other hand, in a homozygous (−/−) individual, uniform fluorescence is shown because the pancreas is constructed by a cell that is completely derived from the ES cell. Using such characteristics, it is possible to conveniently examine which genotype a target organ or a cell constituting the target organ has with respect to the Pdx1 gene. If unmarked SCNT cells are used, the cells cannot be distinguished from the embryos of the host when used in the production of chimera, and it cannot be discriminated whether the complementation of the organ has been achieved. Therefore, a fluorescent dye can be introduced into the SCNT cell line, thereby being capable of carrying out an experiment with the same protocol as above. By using the cell such as described above, it is possible to produce an organ with the same protocol as the case of using the SCNT cell, and to clarify the origin.

(Formation of Kidney)

The formation of a kidney can be investigated by performing macroscopic or microscopic morphological analysis, gene expression analysis, and the like, using methods, such as visual inspection, microscopic observation after staining, and observation using fluorescence.

For example, by performing visual inspection, the actual presence or absence of the organ, and features of the organ, such as the external appearance, can be investigated. Together with such a macroscopic morphological analysis, a tissue obtained after general tissue staining, such as hematoxylin-eosin staining, may be observed microscopically using a microscope. Such microscopic observation allows investigations to be performed, even on various concrete cellular compositions within the kidney.

Furthermore, the gene expression analysis using fluorescence in such a way as to emit fluorescence according to conditions may also be performed. For example, the above-described Sall1 gene knockout mouse has the following characteristics. The fluorescence intensity is low when the deficiency of the Sall1 gene is in the homozygous state (Sall1 (−/−)) where GFP fluorescence occurs from both alleles, compared to the case of fluorescence when the deficiency of the Sall1 gene is in a heterozygous state (Sall1 (+/−)) where fluorescence occurs only in one allele. Using such characteristics, it is possible to conveniently examine which genotype a target organ or a cell constituting the target organ has with respect to the Sall1 gene. If unmarked SCNT cells are used, the cells cannot be distinguished from the embryos of the host when used in the production of chimera, and it cannot be discriminated whether the complementation of the organ has been achieved. Therefore, a fluorescent dye can be introduced into the SCNT cell line to thereby clarify the origin.

(Formation of Hair)

The formation of a hair can be investigated by performing macroscopic or microscopic morphological analysis, gene expression analysis, and the like, using methods, such as visual inspection and observation using fluorescence.

For example, by performing visual inspection, the actual presence or absence of a hair, and features of the hair, such as the external appearance, can be investigated. Together with such a macroscopic morphological analysis, a tissue obtained after general tissue staining, such as hematoxylin-eosin staining, may be observed microscopically using a microscope. Such microscopic observation allows investigations to be performed, even on various concrete cellular compositions within the hair.

Furthermore, the gene expression analysis using fluorescence in such a way as to emit fluorescence according to conditions may also be performed. For example, in the case of the above-described nude mouse, because of strong self-fluorescence of hair, it is very difficult to determine whether the produced hair is derived from the nude mouse or from the SCNT cell with the naked eye under a fluorescent microscope. However, the observation can also be performed by means for appropriately observing the fluorescence. Using such characteristics, it is possible to conveniently examine which genotype a target organ or a cell constituting the target organ has. If unmarked SCNT cells are used, the cells cannot be distinguished from the embryos of the host when used in the production of chimera, and it cannot be discriminated whether the complementation of the organ has been achieved. Therefore, a fluorescent dye can be introduced into the SCNT cell line, thereby being capable of carrying out an experiment with the same protocol as above. By using such cells as described above, it is possible to produce an organ with the same protocol as the case of using the SCNT cell, and to clarify the origin.

(Formation of Thymus)

The formation of a thymus can be investigated by performing macroscopic or microscopic morphological analysis, gene expression analysis, and the like, using methods, such as visual inspection, microphotographs, FACS, and observation using fluorescence.

For example, by performing visual inspection, the actual presence or absence of the organ, and features of the organ, such as the external appearance, can be investigated. Together with such a macroscopic morphological analysis, a tissue obtained after general tissue staining, such as hematoxylin-eosin staining, may be observed microscopically using a microscope. Such microscopic observation allows investigations to be performed, even on various concrete cellular compositions within the thymus.

Furthermore, the gene expression analysis using fluorescence in such a way as to emit fluorescence according to conditions may also be performed. For example, the above-described nude mouse has the following characteristics. The nude mouse does not conventionally have thymus, but this does not affect the survival. Accordingly, the nude mouse is born naturally without the thymus and survives. If a fluorescent-labeled SCNT cell is injected thereinto by blastocyst complementation, a large number of individuals in which the contribution of the SCNT cell is confirmed have the thymus showing fluorescence. Using such characteristics, it is possible to conveniently examine which genotype a target organ or a cell constituting the target organ has.

A target organ obtained in accordance with embodiments herein may be characterized by being completely derived from the second mammal.

The present disclosure also provides a mammal produced by a method in accordance with embodiments herein. Furthermore, the present disclosure also provides use of a non-human first mammal having an abnormality associated with a lack of development of a target organ in a development stage, for production of the target organ.

(Points to Remember when Using Various Animals)

The cases of using animals other than a mouse can be performed by applying a technique described in Examples herein upon paying attention to the following points. For example, regarding the production of a chimera in other species of animals, specifically in species other than mice, there are more reports of chimeras into which an embryo or an inner cell mass, which is a part of an embryo and is an origin of an ES cell, is injected, than reports of establishment of pluripotent stem cells having an ability to form a chimera (rat: (Mayer, J. R. Jr. & Fretz, H. I. The culture of preimplantation rat embryos and the production of allophenic rats. J. Reprod. Fertil. 39, 1-10 (1974)); cattle: (Brem, G. et al. Production of cattle chimerae through embryo microsurgery. Theriogenology. 23, 182 (1985)); pig: (Kashiwazaki N et al., Production of chimeric pigs by the blastocyst injection method, Vet. Rec. 130, 186-187 (1992)). However, even when a chimera into which an inner cell mass is injected is used, the method described herein may be applied. By using an inner cell mass as described above, it is substantially possible to complement a deficient organ of a defected animal. In other words, for example, the above-described cells are each cultivated to grow into a blastocyst in vitro, a portion of inner cell mass is physically separated from thus obtained blastocyst, and then, the portion may be injected into a blastocyst. A chimeric embryo can be produced by agglutinating the 8 cell-stage ones or morulas in mid-course.

(General Techniques)

Molecular biological, biochemical, and microbiological techniques used herein are well known and commonly used in the art, and are disclosed in, for example: Sambrook J. et al. (1989). Molecular Cloning: A Laboratory Manual, Cold Spring. Harbor, and its 3rd Ed. (2001); Ausubel, F. M. (1987). Current Protocols in Molecular Biology, Greene Pub. Associates and Wiley-Interscience; Ausubel, F. M. (1989). Short Protocols in Molecular Biology: A Compendium of Methods from Current Protocols in Molecular Biology, Greene Pub. Associates and Wiley-Interscience; Innis, M. A. (1990). PCR Protocols: A Guide to Methods and Applications, Academic Press; Ausubel, F. M. (1992). Short Protocols in Molecular Biology: A Compendium of Methods from Current Protocols in Molecular Biology, Greene Pub. Associates; Ausubel, F. M. (1995). Short Protocols in Molecular Biology: A Compendium of Methods from Current Protocols in Molecular Biology, Greene Pub. Associates; Innis, M. A. et al. (1995). PCR Strategies, Academic Press; Ausubel, F. M. (1999). Short Protocols in Molecular Biology: A Compendium of Methods from Current Protocols in Molecular Biology, Wiley, and annual updates; Sninsky, J. J. et al. (1999). PCR Applications: Protocols for Functional Genomics, Academic Press; separate-volume laboratory medicine “Experimental technique for gene transfer & expression analysis” Yodosha, 1997; and so on. These documents related to the present description are incorporated herein by reference.

A DNA synthesis technique and nucleic acid chemistry for producing an artificially synthesized gene are disclosed in, for example: Gait, M. J. (1985). Oligonucleotide Synthesis: A Practical Approach, IRL Press; Gait, M. J. (1990). Oligonucleotide Synthesis: A Practical Approach, IRL Press; Eckstein, F. (1991). Oligonucleotides and Analogues: A Practical Approach, IRL Press; Adams, R. L. et al. (1992). The Biochemistry of the Nucleic Acids, Chapman & Hall; Shabarova, Z. et al. (1994). Advanced Organic Chemistry of Nucleic Acids, Weinheim; Blackburn, G. M. et al. (1996). Nucleic Acids in Chemistry and Biology, Oxford University Press; Hermanson, G. T. (1996). Bioconjugate Techniques, Academic Press; and so on. The parts of these documents related to the present description are incorporated herein by reference.

Reference documents cited herein, such as science documents, patents, and patent applications, are incorporated herein by reference in their entirety to an extent that each of which is specifically described.

The preferred embodiments have been described for easy understanding of the present disclosure. Hereinafter, the present disclosure will be described based on examples; however, the above description and the following examples are provided only for exemplary purposes and are not provided for the purpose of limiting the present disclosure. Therefore, the scope of the present disclosure is not limited to the embodiments or examples which are specifically described herein, and is limited only by the claims.

The particular embodiments disclosed above are illustrative only, as the disclosure may be modified and practiced in different but equivalent manners apparent to those skilled in the art having the benefit of the teachings herein. For example, the process steps set forth above may be performed in a different order. Furthermore, no limitations are intended to the details of construction or design herein shown, other than as described in the claims below. It is, therefore, evident that the particular embodiments disclosed above may be altered or modified and all such variations are considered within the scope and spirit of the disclosure. Accordingly, the protection sought herein is as set forth in the claims below.

What is claimed is:

1. A method for producing a target organ in a living body of a non-human first mammal having an abnormality associated with a lack of development of the target organ in a development stage, the target organ produced being derived from a second mammal that is an individual different from the non-human first mammal, the method comprising: a) preparing a somatic cell nuclear transfer cell (SCNT cell) derived from the second mammal; b) transplanting the SCNT cell into a blastocyst of the non-human first mammal, to yield a chimeric embryo; c) developing the chimeric embryo in a uterus of a non-human third mammal to obtain at least one offspring comprising the target organ; and d) obtaining the target organ from the at least one offspring.

2. The method according to claim 1, wherein the second mammal is a human, a rat, or a mouse.

3. The method according to claim 1, wherein the second mammal is a human.

4. The method according to claim 1, wherein the organ to be produced is selected from a pancreas, a kidney, a thymus, and a hair.

5. The method according to claim 1, wherein the non-human first mammal is a mouse, a rat, a monkey, or a pig.

6. The method according to claim 5, wherein the non-human first mammal is a mouse, and the mouse is one or more of a Sall 1 knockout mouse, a Pdx1-Hes1 transgenic mouse, a Pdx1 knockout mouse, and a nude mouse.

7. The method according to claim 1, wherein the blastocyst is prepared from an oocyte of the non-human first mammal in which one or more genes associated with the abnormality have been silenced by a CRISPR technique and a sperm of the non-human first mammal in which one or more genes associated with the abnormality have been silenced by a CRISPR technique.

8. The method according to claim 1, wherein the target organ is completely derived from the second mammal.

9. The method according to claim 1, wherein the second mammal and the non-human first mammal are in a xenogeneic relationship.

10. A non-human first mammal having an abnormality associated with a lack of development of a target organ in a development stage, the mammal being produced by a method comprising: a) preparing an SCNT cell derived from a second mammal that is an individual different from the non-human first mammal; b) transplanting the SCNT cell into a blastocyst of the non-human first mammal, to yield a chimeric embryo; and c) developing the chimeric embryo in a uterus of a non-human third mammal to obtain at least one offspring comprising the target organ.

11. A kit for producing a target organ, the kit comprising: A) a non-human first mammal having an abnormality associated with a lack of development of the target organ in a development stage; and B) an SCNT cell derived from a second mammal that is an individual different from the non-human first mammal.

12. A method for producing one or more of a target organ and a target body part, the method comprising:
providing a non-human animal which includes a deficiency-responsible gene coding for a factor which causes a deficiency of one or more of an organ and a body part and gives one or more of no possibility of survival and difficulty in survival if the factor functions, and in which the one or more of an organ and a body part is complemented by blastocyst complementation, the deficiency-responsible gene coding for a factor which causes a deficiency of the one or more of a target organ and a target body part;
growing an ovum obtained from the non-human animal into a blastocyst;
introducing a target somatic cell nuclear transfer cell (SCNT cell) into the blastocyst so as to produce a chimeric blastocyst, the target SCNT cell having a desired genome capable of complementing a deficiency caused by the deficiency-responsible gene;
producing an individual from the chimeric blastocyst; and
obtaining the one or more of a target organ and a target body part from the individual.

13. The method according to claim 12, wherein producing the individual further comprises developing the chimeric blastocyst in a uterus of a non-human third mammal to obtain at least one offspring comprising the target organ, and obtaining the target organ from the at least one offspring.

14. The method according to claim 12, wherein the target SCNT cell is derived from a human.

15. The method according to claim 12, wherein the one or more of a target organ and a target body part is selected from a pancreas, a kidney, a thymus, and a hair.

16. The method according to claim 12, wherein the non-human animal is a mouse, a rat, a monkey, or a pig.

17. The method according to claim 16, wherein the non-human animal is a mouse, and the mouse is one or more of a Sall 1 knockout mouse, a Pdx1 knockout mouse, a Pdx1-Hes1 transgenic mouse, and a nude mouse.

18. The method according to claim 12, wherein the one or more of a target organ and a target body part is completely derived from the target SCNT cell.

19. The method according to claim 12, further comprising preparing the SCNT cell, wherein the SCNT cell is derived from a second mammal.

20. The method according to claim 19, wherein the second mammal and the non-human animal are in a xenogeneic relationship.

21. The method according to claim 12, wherein the blastocyst is prepared from an oocyte of the non-human first mammal in which one or more genes, including the deficiency-responsible gene, have been silenced by a CRISPR technique and a sperm of the non-human first mammal in which one or more genes, including the deficiency-responsible gene, have been silenced by a CRISPR technique.

22. A kit for producing one or more of a target organ and a target body part, the kit comprising: A) a non-human animal which includes a gene coding for a factor which causes a deficiency of one or more of an organ and a body part and gives one or more of no possibility of survival and difficulty in survival if the factor functions, and in which the one or more of an organ and a body part is complemented by complement; and B) an SCNT cell derived from a second mammal that is an individual different from the non-human animal.

23. The kit according to claim 22, wherein the non-human animal and the second mammal are in a xenogeneic relationship.
